(19) United States
(12) Patent Application Publication
(10) Pub. No.: US 2026/0199599 A1
(43) Pub. Date: Jul. 16, 2026

(54) DEDICATED CONSUMABLE SETS WITH FLOW RATE SENSORS

(71) Applicant: ICU Medical, Inc., San Clemente, CA (US)

(72) Inventors: James Jacobson, Lindenhurst, IL (US); Robert Butterfield, Poway, CA (US); Robert Hlinsky, Glenview, IL (US); Marshall E. Fryman, Libertyville, IL (US)

(21) Appl. No.: 19/135,905

(22) PCT Filed: Dec. 8, 2023
(86) PCT No.: PCT/US2023/083038

§ 371 Date: Jun. 5, 2025

Related U.S. Application Data
(60) Provisional application No. 63/431,421, filed on Dec. 9, 2022.
(60) Provisional application No. 63/435,996, filed on Dec. 29, 2022.

(51) Int. Cl. A61M 5/168; A61M 5/145

(52) U.S. Cl. CPC A61M 5/16886; A61M 5/1452; A61M 2205/3334; A61M 2205/50; A61M 2205/60

(57) ABSTRACT

The present disclosure describes dedicated consumable sets for use with syringe pumps. A dedicated consumable set for use with a syringe pump may include a housing configured to receive a flow rate sensor communicatively coupled to the syringe pump and configured to measure flow rate of an infusate, and an optional RFID tag fixedly coupled to the housing and configured to communicate information about the dedicated consumable set to the syringe pump. The flow rate sensor can be further configured to detect the presence of an occlusion and communicate detection of the occlusion to the syringe pump. Advantages of the present disclosure include improvements to syringe pump start-up time, syringe swap time, and syringe pump occlusion detection and resolution times.

### CROSS REFERENCE TO RELATED APPLICATIONS

The present application claims the benefit of U.S. Provisional Application No. 63/435,996, filed Dec. 29, 2022 and U.S. Provisional Application No. 63/431,421, filed Dec. 9, 2022, the disclosures of which are hereby incorporated by reference.

### TECHNICAL FIELD

The present disclosure generally pertains to dedicated consumable sets offering advanced functionality when used with infusion pumps. The present disclosure particularly pertains to dedicated consumable sets with flow rate sensors and/or RFID tags, for use with infusion pumps.

### BACKGROUND

Infusion pumps are extremely useful medical devices for providing prescribed fluids, drugs, and other therapies (collectively, “infusates”) to patients in controlled amounts. For example, medications such as antibiotics, chemotherapy drugs, vasoactives, insulin, blood products, and pain relievers are commonly delivered to patients via infusion pumps, as are nutrients and other supplements. Infusion pumps have been used in hospitals, nursing homes, and in other short-term and long-term medical facilities, as well as for in-home care. Infusion pumps can be particularly useful for the delivery of medical therapies requiring an extended period of time for their administration. There are many types of infusion pumps, including large volume, patient-controlled analgesia (PCA), elastomeric, syringe (syringe driver), enteral, and insulin pumps. Infusion pumps are typically useful in various routes of medication delivery, including intravenously, intra-arterially, subcutaneously, intraperitoneally, intraosseous, intraportal, in close proximity to nerves, and into an intraoperative site, epidural space or subarachnoid space.

Syringe pumps have several desirable characteristics and are generally perceived as the most precise and accurate acute care infusion pumps available. Syringe pumps may support lower flow rates than large volume pumps or ambulatory pumps, sometimes as low as 0.01 milliliters/hour (mL/hr) with appropriately-sized small syringes. Unlike large volume and ambulatory pumps that utilize proprietary or dedicated consumables, syringe pumps typically accommodate wide ranges of commonly used or “off-the-shelf” syringe brands and sizes that are typically coupled with non-proprietary extension sets for delivering infusates to patients.

Some syringe pumps can suffer from performance limitations, particularly at low flow rates (below 5 mL/hr and particularly below 0.1 mL/hr), including but not limited to long times to reach target flow rates, inconsistent flow profiles during infusate delivery, long times to detect an occlusion, and risk of inadvertently delivering a bolus or allowing retrograde flow due to varying external pressure. Further, dynamic flow inaccuracies may arise from changes in backpressure due to multiple pumps being added to, or removed from, the same infusion line.

Off the shelf or non-dedicated tubing consumables have traditionally supported only fluid routing between a syringe pump and a patient access device (such as a catheter) for delivery of fluid. While the traditional use of simple off-the-shelf or non-dedicated tubing sets provides clinicians with flexibility in selecting different tubing set lengths, materials, priming volumes and compliance characteristics from among numerous vendors, there is a need for more sophisticated consumable tubing sets to integrate more fully with pump operation. Dedicated tubing sets will provide improved operation including accelerated start-up time and occlusion detection time, and the ability to address other known operating issues with syringe pumps.

Upon initiation of a programmed infusion via a syringe pump, there can be a substantial delay before the programmed rate is achieved, particularly at low rates and with large volume syringes. This is due to mechanical slack in the pump system that must be absorbed by the syringe pump mechanism before the mechanism starts to effectively drive the syringe plunger into the syringe barrel. Once the pump mechanism has engaged with the syringe plunger and started forcing the syringe plunger into the syringe barrel, it takes further time before the system is operating at the desired programmed rate as compliance of the pump, syringe, and tubing set absorbs incremental fluid volume until a steady state flow rate is achieved. At low and very low rates, and/or with large volume syringes, it can typically take hours for the system to reach a target flow rate. Aspects of this disclosure greatly accelerate the start-up time of syringe pumps, particularly at low and very low rates.

During operation of a syringe pump delivering infusates, an occlusion might occur when the fluid line is clamped, blocked or otherwise impeded, as when for example the infusion line tubing is kinked or the catheter becomes blocked due to formation of thrombotic or non-thrombotic blockage. If the occlusion is not noticed by a care provider or detected by the syringe pump, the patient likely would not receive the prescribed medication leading to potentially serious consequences. At relatively low flow rates, the amount of time to detect an occlusion may be unacceptably long as intended and commanded forward progression of the plunger does not deliver fluid to the patient, but rather introduces fluid which is stored as incremental volume within the compliances of the pump, the syringe, or the set.

Some current syringe pumps include a force sensor in the plunger driver head which indirectly senses the fluid pressure by measurement of syringe plunger force. When the force or rate of change of force detected by that sensor exceeds predetermined thresholds, a processor monitoring that signal generates an indication that an occlusion has possibly occurred or is possibly occurring presently and may pause movement of the driver head. Since syringe pumps are typically capable of accommodating a wide range of syringe diameters or sizes (e.g., 1 mL through 100 mL capacities) and exhibit a wide range of stopper friction forces, the driver head and force sensor may experience varying occlusion forces depending upon the syringe being used leading to varying accuracy and responsiveness overall in the pump's occlusion sensing system. Because syringe pumps typically need to accommodate a wide range of syringe sizes, the accuracy of detecting an occlusion at low flow rates and/or with smaller syringes may be decreased. Aspects of the current disclosure greatly reduce the time required for the pump system to recognize an occlusion, particularly at low rates.

While valuable improvements to syringe pump constructions, configurations, and operations have been and continue to be made, there remains a need for improvements to consumable tubing sets themselves and associated components for their uses in conjunction with syringe pumps. The present disclosure addresses these concerns.

### SUMMARY

Embodiments described or otherwise contemplated herein substantially provide the advantages of improving ease of use, operation, accuracy, and patient safety in the delivery of infusates, among other advantages.

In embodiments, a dedicated consumable set for use with a syringe pump may comprise a housing configured to receive a flow rate sensor communicatively coupled to the syringe pump and configured to measure flow rate of an infusate, and an RFID tag fixedly coupled to the housing and configured to communicate information about the dedicated consumable set to the syringe pump. The flow rate sensor may be further configured to quantify flow rate, particularly during start-up, and detect the presence of an occlusion and communicate detection of the occlusion to the syringe pump.

In features and advantages of embodiments, the present disclosure provides the following improvements over prior approaches: the ability to provide short and long-term accuracy across a full range of syringe pump operations independent of environmental conditions such as backpressure, temperature, and flow variations;
the ability to quickly achieve a target flow rate after syringe pump startup
the ability to quickly recognize during delivery if an incorrect syringe size has been selected by a clinician during pump programming (e.g., 1 cc syringe selected instead of 3 cc syringe);
the ability to monitor flow constancy and recognize perturbations such as the “stick-slip” phenomena which will allow the ability to modify pumping behavior on-the-fly to return to more continuous flow;
the ability to rapidly recognize changes in flow delivery, potentially due to dynamics of other pumps connected to the same patient delivery line; and
the ability to compensate changes to syringe dimensions or physical parameters (e.g., stiffness) via flow rate sensing, which may include the ability to recognize an incorrectly selected syringe.

With regard to changes in flow delivery as aforementioned, it is to be appreciated and understood that multiple syringe pumps and large volume pumps may be utilized together to deliver an infusate or infusates through the same catheter or catheter lumen. However, the pumps may be operating at different infusion rates. So until fluid dynamics of such a system stabilize, there is the potential that a low infusion rate pump may not be accurately infusing because other higher rate infusion pumps, that create backpressure, may deleteriously impact fluid delivery accuracy.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is a perspective view of an example syringe pump for use with embodiments of the disclosure.

FIG. 2 is a schematic view of a syringe and dedicated tubing set assembly, according to an embodiment of the disclosure.

FIG. 3 is a graph of flow rate over time of a 50-milliliter syringe comparing a system of the present disclosure versus a prior art pressure detection system, demonstrating accelerated occlusion detection time, according to an embodiment.

FIG. 4 is a graph of flow rate over time of a 5-milliliter syringe comparing a system of the present disclosure versus a prior art pressure detection system, demonstrating accelerated occlusion detection time, according to an embodiment.

FIG. 5 is a graph of flow rate over time of a 5-milliliter syringe comparing a system of the present disclosure versus a prior art pressure detection system, demonstrating accelerated occlusion detection time, according to an embodiment.

FIG. 6 is a table summarizing flow rate data for preliminary time to occlusion detection for several syringe configurations, according to an embodiment.

FIG. 7 is a graph of flow rate over time of a 50/60-milliliter syringe, demonstrating cold load startup delay, according to an embodiment.

FIG. 8 is a graph of flow rate over time of a 50/60-milliliter syringe, demonstrating delay in reaching target flow rate with acceleration, according to an embodiment.

FIG. 9 is a graph of flow rate over time of a 50/60-milliliter syringe, demonstrating cold load startup delay and highlighting an accelerated phase region, according to an embodiment.

FIG. 10A is a graph of flow rate over time of a 50/60-milliliter syringe, demonstrating cold load startup delay with and without acceleration for an intended flow rate of 1.0 milliliters per hour, according to an embodiment.

FIG. 10B is a graph of flow rate over time of a 50/60-milliliter syringe, demonstrating cold load startup delay with and without acceleration for a flow rate of 0.1 milliliters per hour, according to an embodiment.

While various embodiments are amenable to various modifications and alternative forms, specifics thereof have been shown by way of example in the drawings and will be described in detail. It should be understood, however, that the intention is not to limit the claimed subject matter to the particular embodiments described. On the contrary, the intention is to cover all modifications, equivalents, and alternatives falling within the spirit and scope of the subject matter as defined by the claims.

### DETAILED DESCRIPTION OF THE DRAWINGS

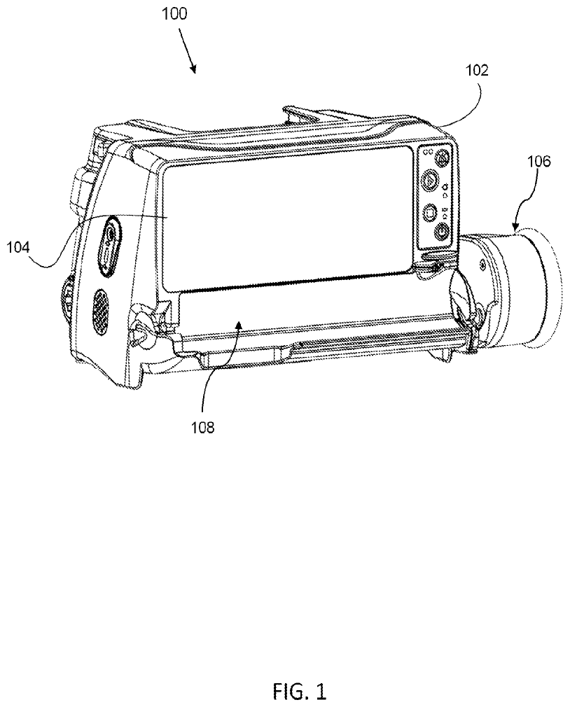

Referring to FIG. 1, an example of a syringe pump 100 is depicted for use with embodiments of the present disclosure. Syringe pump 100 can include a housing 102, user interface 104, a syringe drive assembly 106, and a syringe receptacle 108.

The syringe drive assembly 106 can be employed for controlling delivery of a prescribed amount or dose of an infusate from a syringe that has been installed in the pump 100 (not illustrated in FIG. 1) to a patient by mechanically advancing a plunger in the syringe to deliver the infusate at a controlled rate through an infusion line fluidly connected to the syringe. In an example, a motor within pump 100 rotates a lead screw which, in turn, causes a plunger driver head assembly of the syringe drive assembly 106 to move in a direction toward the syringe receptacle 108. This movement then pushes the plunger within a barrel of the syringe located within the receptacle 108, where the barrel is held substantially in place. Moving the syringe plunger forward acts to displace a volume of infusate in the syringe outwardly from the syringe, into the infusion line, and ultimately to the patient.

In the example pump 100 of FIG. 1, the syringe receptacle 108 provides a cavity extending across the front of the syringe pump 100 such that a syringe installed therein is readily and sustainably visible. The syringe receptacle 108 is shaped and sized to accept installation of syringes of various sizes and brands therein for delivery of infusates.

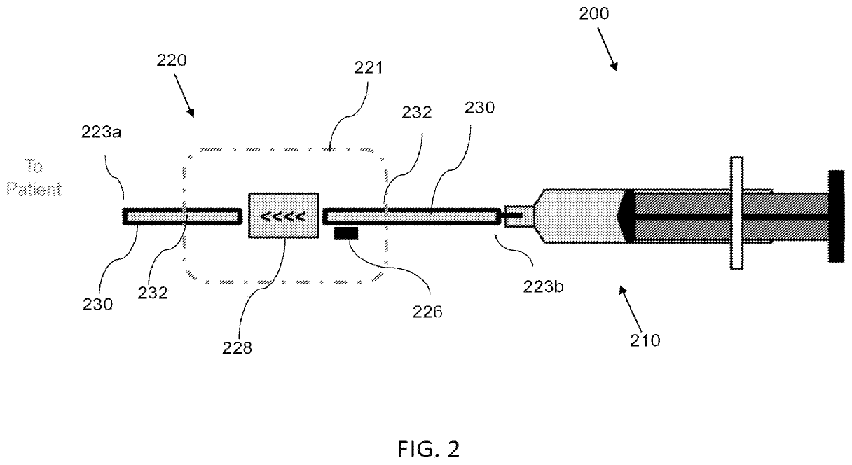

Referring now to FIG. 2, in an embodiment, a syringe and dedicated tubing set assembly 200 for use with a syringe pump 100 includes a syringe 210 connected to a dedicated consumable set 220, the dedicated consumable set 220 including an optional housing 221, an optional radio-frequency identification (RFID) tag 226, a flow rate sensor 228, tubing 230, and tubing connectors 232.

RFID tag 226 can be configured as active or passive. RFID tag 226 may include identifying information pertaining to the set, such as tubing length, tubing diameter, priming volume, date of manufacture, type of flow rate sensor 228, or other information as desired (e.g., enteral, intravenous, or subcutaneous epidural delivery route identification). RFID tag 226 may be communicable with syringe pump 100 or other devices, such as an RFID tag reader operable by a clinician.

Flow rate sensor 228 can be configured to measure infusate flow rates which assists rapid occlusion detection at very low flow rates and further improves the functionality and control of syringe and dedicated set assembly 200. Flow rate sensor 228 can be positioned between distal and proximal ends 223a,b of dedicated consumable set 220. Flow rate sensor 228 may comprise any sensor suitable for measuring flow rate of a syringe and dedicated set assembly 200 including, but not limited to, a thermal-based sensor, a Coriolis sensor, and a micro-fluidic sensor. In embodiments, syringe and dedicated set assembly 200 may comprise more than one flow rate sensor 228 of any suitable type or various types, positioned at, for example, various locations along tubing 230. Advantages of incorporating a flow rate sensor 228 into dedicated consumable set 220 include, for example, an ability to enable rapid achievement or programmed flow rate, an ability to rapidly recognize changes in flow delivery potentially due to dynamics of other pumps connected to the same patient delivery line, a potential to sense line disconnections, and minimizing potential occurrence of an unintended bolus due to head height changes.

Tubing 230 is configured to attach to a tubing connector 232 positioned on each end of dedicated consumable set 220 to create a pathway for the flow of infusate from syringe 210 through dedicated consumable set 220 and to a patient. Additional sections of tubing 230 may also be provided as desired, for example between flow rate sensor 228 and RFID tag 226. In embodiments, tubing 230 may be provided between an outlet of syringe 210 and an inlet of dedicated consumable set 220. Tubing 230 may also be provided between an outlet of dedicated consumable set 220 and a patient access device, such as a catheter. Tubing 230 may be manufactured from a medical-grade plastic including, but not limited to, silicone, polyethylene, polyurethane, or polyvinyl chloride. Tubing connectors 232 may be manufactured from a medical grade plastic including, but not limited to, polycarbonate.

In embodiments, dedicated consumable set 220 may further include particulate or air elimination filters and light-resistant tubing or housing (both not depicted). Filters would be added to eliminate particulate, bacteria, or virus accumulation in dedicated consumable set 220. Light-resistant components of dedicated consumable set would help to maintain safety and/or efficacy of infusates that are susceptible to degradation from light.

In embodiments, a simple occlusion detection algorithm with long data averaging windows and a simple alarm threshold may be used with syringe and dedicated set assembly 200, for example by means including monitoring flow rate through flow rate sensor 228.

In an example “fast start”, “rapid start-up”, or “accelerated start-up” implementation, flow may commence at a rate substantially greater than the programmed rate and then decelerate over time until the programmed rate is reached. This allows the programmed rate to be achieved quickly and without risk of overshooting to an unacceptable rate. Flow rate sensor 228 in the line may allow such implementations to operate more efficiently and accurately based at least in part on measurement of flow rate.

In embodiments, a simple accelerated start-up algorithm utilizing an increased initial mechanical drive speed while monitoring flow rate to recognize removal of mechanical slack from the system and incremental absorption of system compliance could be used to accelerate start-up, with reduction of the mechanism to programmed rate after a threshold flow rate profile is attained. Alternatively, a more sophisticated algorithm of an accelerate mechanism drive rate followed by discrete deceleration steps could be applied to accelerate the start-up of syringe pump 100. For example, algorithms disclosed in U.S. Pat. No. 11,179,515, incorporated herein in its entirety, could be used as may be applicable or suitable for, or with, at least one flow rate sensor.

In embodiments, flow rate sensor 228 can be registered to syringe pump 100 mechanically and be electrically couplable to the syringe pump 100, or the flow rate sensor 228 can be communicatively couplable to the syringe pump 100 and also located in close proximity thereto. In embodiments, flow rate sensor 228 can be powered inductively or with a unique or otherwise suitable battery or electrical power device.

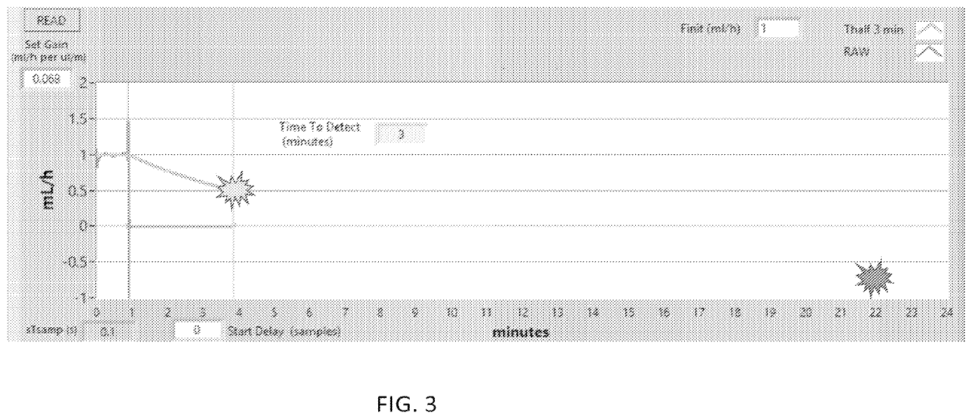

Referring now generally to FIGS. 3-5, graphs of flow rate over time of different-sized syringes comparing systems of the present disclosure versus conventional pressure detection systems (with or without an inline pressure-sensing diaphragm, “disk”, or similarly functioning component, such as described in, for example, U.S. Pat. No. 4,398,542) are depicted, demonstrating accelerated or shortened accurate occlusion detection time even with a simple, non-optimized control algorithm.

FIG. 3 specifically depicts an example of flow rate over time for a 50-milliliter syringe with a steady state flow rate of 1.0 mL/hr with an applied occlusion compared to a conventional pressure detection system with an inline pressure-sensing disc at a pressure setting of 50 mm Hg. Time to occlusion detection for a flow rate sensor embodiment of the present disclosure is 3 minutes compared to approximately 22 minutes for the conventional pressure detection system. This demonstrated improvement was obtained using a 3-minute data averaging window and a threshold of 50% of the target flow rate, though more sophisticated algorithms could be employed. Direct flow rate is shown, as is the filtered date.

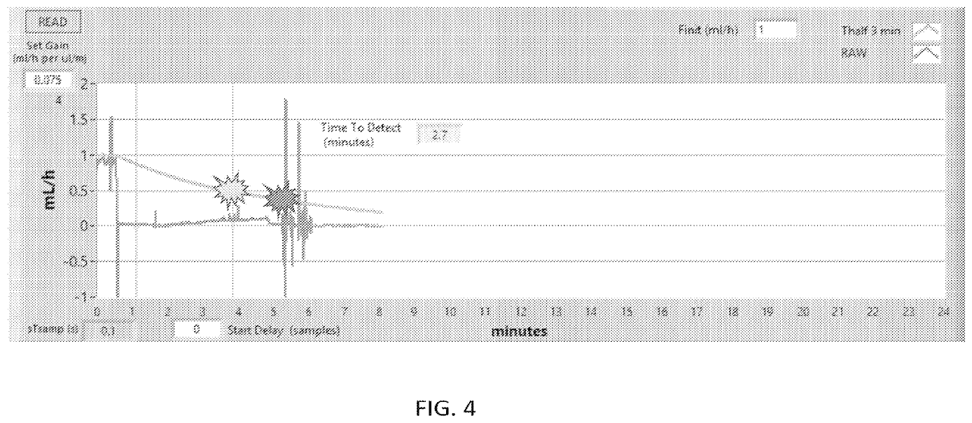

FIG. 4 specifically depicts an example of flow rate over time for a 5-milliliter syringe with a steady state flow rate of 1.0 mL/hr and an applied occlusion compared to a conventional pressure detection system with an inline pressure-sensing disc at a pressure setting of 50 mm Hg. Time to occlusion detection for a flow rate sensor embodiment of the present disclosure is 2.7 minutes compared to over 4 minutes for the conventional pressure detection system.

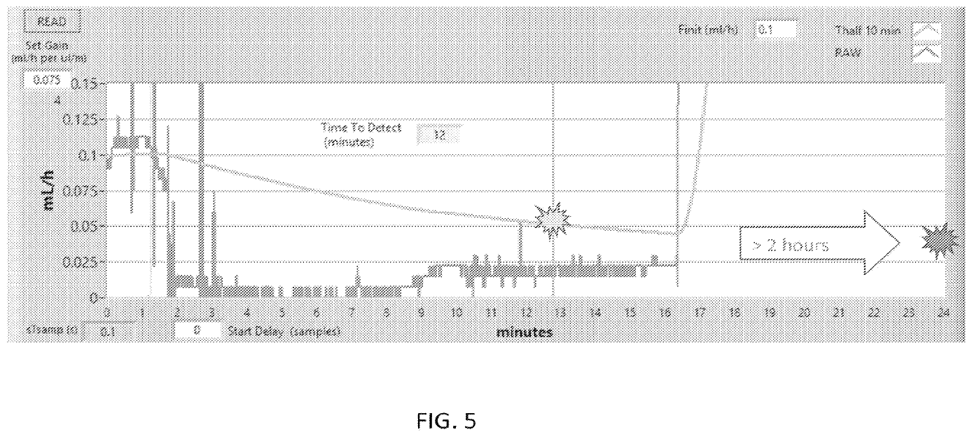

FIG. 5 specifically depicts an example of flow rate over time for a 5-milliliter syringe with a steady state flow rate of 0.1 mL/hr and an applied occlusion compared to a conventional pressure detection system without an inline pressure-sensing disc at a pressure setting of 200 mm Hg. Time to occlusion detection for a flow rate sensor embodiment of the present disclosure is 12 minutes compared to over 120 minutes for the conventional pressure detection system.

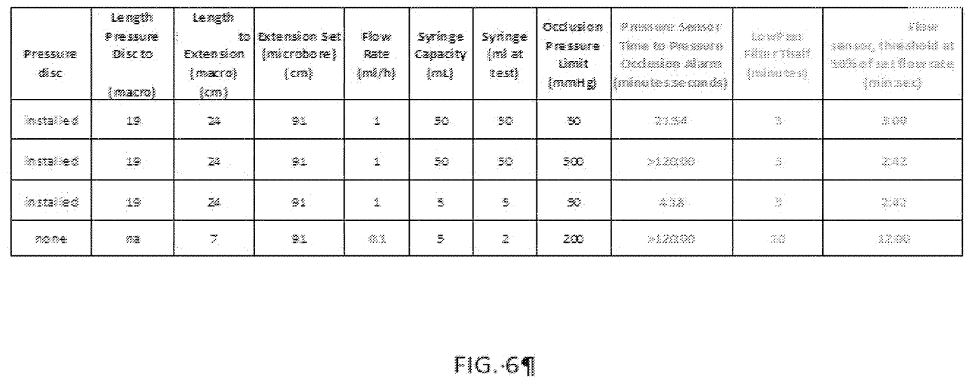

Referring now to FIG. 6, results of time to occlusion detection experiments of FIGS. 3-5 are summarized in tabular form. In all examples, a flow rate sensor embodiment of the present disclosure detected the occlusion in less time compared to the concurrently-tested conventional pressure detection system.

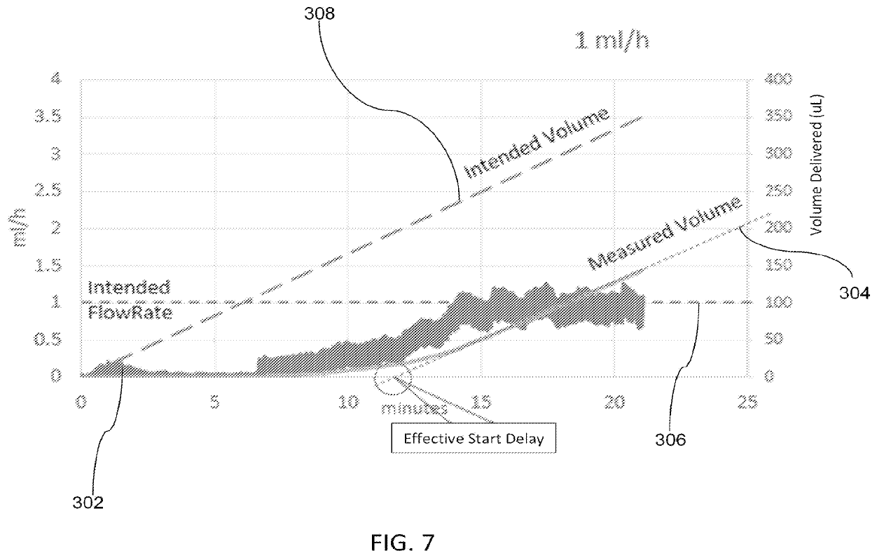

Referring now to FIG. 7, a graph of flow rate over time data 302 for a 50/60-milliliter syringe with an intended flow rate 306 of 1.0 mL/hr using a syringe pump demonstrates cold load startup delay comparing intended flow volume 308 and measured flow volume 304. As used herein, the term “cold load” refers to a syringe pump infusion that starts with a manually primed fluid line but without pump-based priming or any additional running of the pump to reduce mechanical slack and system compliance prior to initiation of the infusion. In this example, the effective start delay is 12 minutes, for cold load startup at the intended flow rate 306.

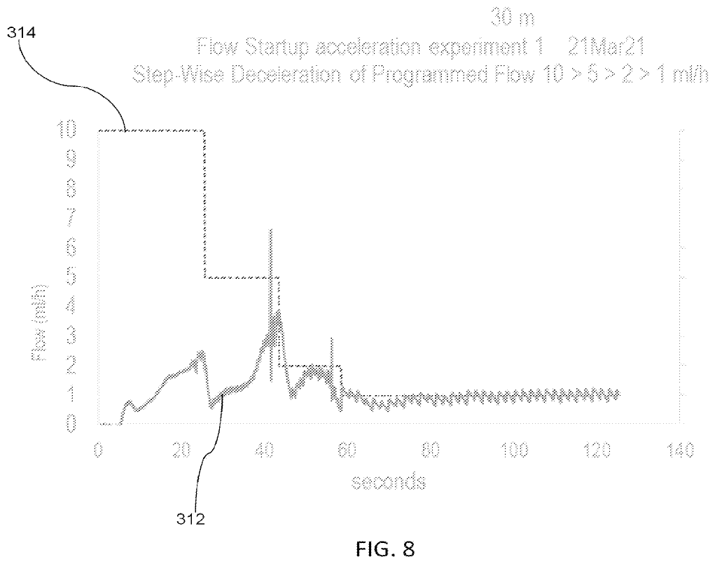

Referring now to FIG. 8, a graph of flow rate over time data 312 for a 50/60-milliliter syringe with an intended flow rate of 1.0 mL/hr using a syringe pump with step-wise deceleration 314 of programmed flow from 10 to 5 to 2 to 1 mL/hr demonstrates a delay in reaching the target flow rate. The experiment started with the syringe pump operating at a target flow rate of 10 mL/hr, followed by a manual reduction to 5 mL/hr when the flow rate sensor detected that the flow rate exceeded 2 mL/hr, followed by a manual reduction to 2 mL/hr when the flow rate sensor detected that the flow rate exceeded 3 mL/hr, ending with a manual reduction to 1 mL/hr when the flow rate sensor detected that the flow rate exceeded 2 mL/hr. In this example, the target flow rate was established in approximately one minute, about twelve times faster than cold load start, after commencing the test with manual ad hoc deceleration guided by flow monitoring.

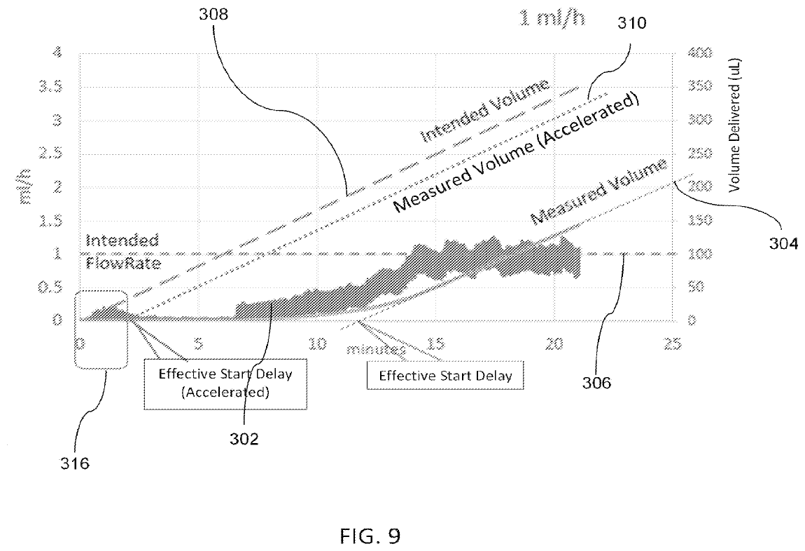

Referring now to FIG. 9, the graph of FIG. 7 is shown with an added volume infused over time curve 310 for the accelerated start-up scenario outlined in FIG. 8. for direct comparison of the non-accelerated 304 and accelerated syringe pump start-ups. Region 316 between 0 and approximately two minutes is also circled. It is noted that an entire accelerated phase shown in FIG. 8 fits within the circled region 316 (i.e., two minutes of accelerated flow with target flow rate being reached within approximately one minute). As depicted in FIG. 9, the effective-start-delay for the non-accelerated flow is about 12 or 13 minutes. By comparison the effective-start-delay for the accelerated flow is less than about 2 minutes. Flow rate sensor embodiments of the present disclosure therefore demonstrate about a seven-fold improvement on startup delay compared to conventional systems.

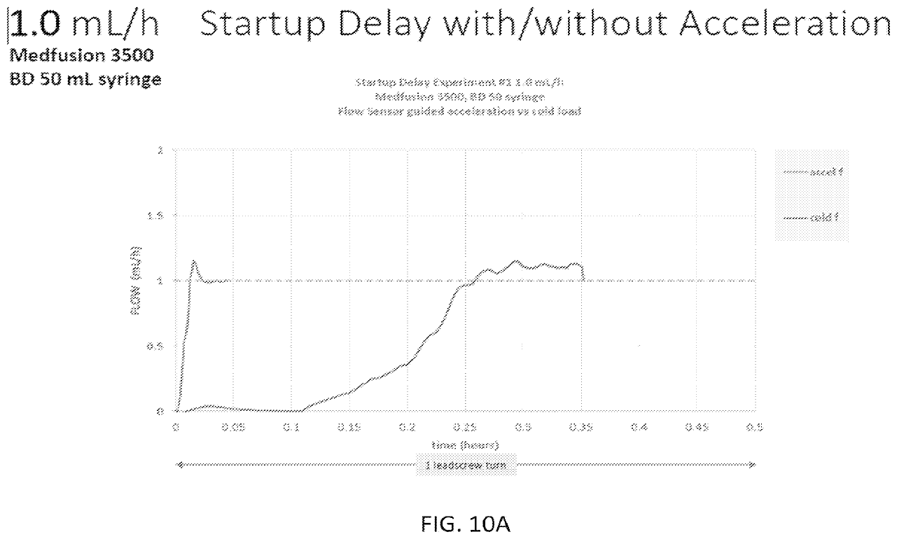

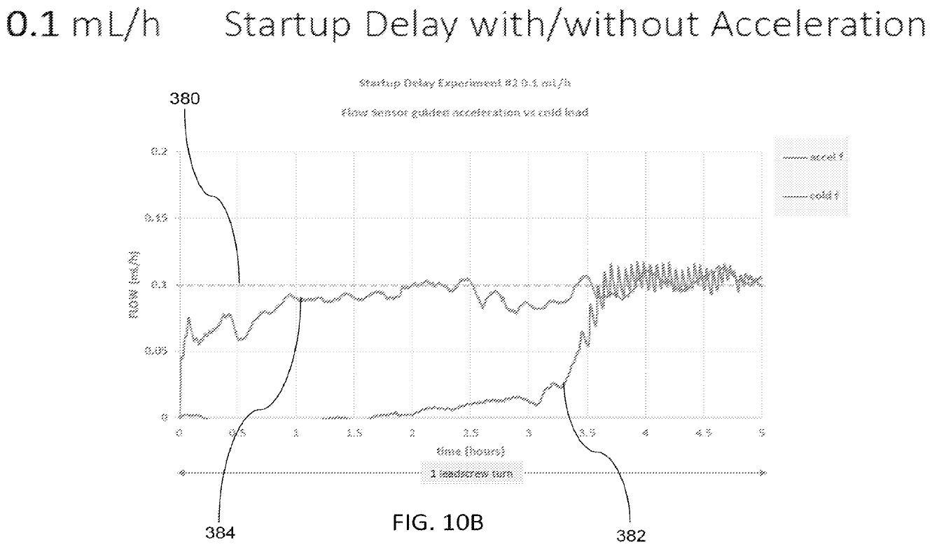

Referring now to FIG. 10A, a graph of flow rate over time of a 50/60-milliliter syringe with an intended flow rate 360 of 1.0 mL/hr using a syringe pump is depicted. This graph compares cold load configuration data 362 to flow rate sensor guided acceleration data 364 and shows that startup delay time is effectively reduced from 13 minutes to one minute with basic manual stepped deceleration. Accordingly, flow rate sensor embodiments of the present disclosure have a potential to significantly outperform conventional systems in startup time and performance.

Referring now to FIG. 10B, a graph of flow rate over time of a 50/60-milliliter syringe with an intended flow rate 380 of 0.1 mL/hr using a syringe pump is depicted. This graph compares cold load configuration data 382 to flow rate sensor guided acceleration data 384 and shows a reduction in startup time from 3.5 hours for a conventional system to less than an hour for flow rate sensor embodiments of the present disclosure. It should be noted that a flow rate of 0.1 mL/hr is below the minimum flow rate value recommended by the syringe pump used in this experiment.

Various embodiments of systems, devices, and methods have been described herein. These embodiments are given only by way of example and are not intended to limit the scope of the claimed subject matter. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed subject matter.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A dedicated consumable set for use with a syringe pump, comprising:
a housing;
a flow rate sensor communicatively coupled to the syringe pump and configured to measure flow rate of an infusate, the flow rate sensor of a type selected from the group consisting of: a thermal sensor, a Coriolis sensor, or a micro-fluidic sensor; and
an RFID tag fixedly coupled to the housing and including identifying information about the dedicated consumable set, wherein the RFID tag is configured to communicate the information to the syringe pump,
wherein the flow rate sensor is configured to provide feedback to the syringe pump to modify an operating parameter of the syringe pump.

2. (canceled)

3. The dedicated consumable set of claim 1, wherein the dedicated consumable set is electrically coupled to the syringe pump.

4. The dedicated consumable set of claim 1, wherein the dedicated consumable set is powered inductively by the syringe pump.

5. The dedicated consumable set of claim 1, wherein the flow rate sensor is configured to provide feedback to the syringe pump to detect an occlusion.

6. The dedicated consumable set of claim 1, wherein the flow rate sensor is configured to provide feedback to the syringe pump to accelerate start-up time.

7. A system for delivering infusate from a syringe, the system comprising:
an infusion pump including a housing, a syringe drive assembly, and a syringe receptacle comprising a cavity extending across a front of the housing; and
an infusion set including:
a flow rate sensor communicatively coupled to the infusion pump and configured to measure flow rate of the infusate;
an RFID tag fixedly coupled to the housing and including identifying information about the dedicated consumable set, wherein the RFID tag is configured to communicate the information to the infusion pump; and
an outlet connected to an infusion line.

8. The system of claim 7, wherein the flow rate sensor is of a type selected from the group consisting of: a thermal sensor, a Coriolis sensor, or a micro-fluidic sensor.

9. The system of claim 7, wherein an operating parameter of the infusion pump is modified based on information from the RFID tag of the infusion set.

10. The system of claim 7, wherein the infusion pump is configured to:
perform a start-up routine wherein the syringe drive assembly is operated at a first, increased speed while monitoring flow rate with the flow rate sensor; and
reduce operation speed of the syringe drive assembly after a threshold flow rate is attained.

11. The system of claim 7, wherein the flow rate sensor is configured to detect backpressure in the infusion set caused by multiple infusion pumps connected to the infusion line.

12. The system of claim 7, wherein the flow rate sensor is configured to detect disconnection from the infusion line.

13. The system of claim 7, wherein the flow rate sensor is configured to minimize occurrence of an unintended bolus by communicating flow rate to the infusion pump such that the infusion pump can modify an operating parameter of the infusion pump to minimize occurrence of the unintended bolus.
